(19) United States
(12) Patent Application Publication
(10) Pub. No.: US 2022/0170029 A1
(43) Pub. Date: Jun. 2, 2022

(54) APPLICATION OF NAT1 GENE AS SCREENING MARKER IN GENETIC TRANSFORMATION OF OOMYCETE

(71) Applicant: Shandong Agricultural University, Tai'an (CN)

(72) Inventors: Qunqing WANG, Tai'an (CN); Xinwei TAN, Tai'an (CN); Shenshen ZOU, Tai'an (CN)

(21) Appl. No.: 17/675,083

(22) Filed: Feb. 18, 2022

Related U.S. Application Data
(63) Continuation of application No. PCT/CN2021/139229, filed on Dec. 17, 2021.

(30) Foreign Application Priority Data
Feb. 3, 2021 (CN) 202110147090.7

(51) Int. Cl. C12N 15/65; C12N 15/80

(52) U.S. Cl. CPC C12N 15/65; C12N 15/80

(57) ABSTRACT

Disclosed is an application of Nat1 gene as a screening marker in the genetic transformation of an oomycete, where the Nat1 gene is employed as a screening marker to screen an oomycete transformant. A nucleotide sequence of the Nat1 gene is shown in SEQ ID NO: 1. This disclosure further provides a Nat1 gene-related biological material, which is a Nat1 gene-containing expression cassette, a recombinant vector containing the Nat1 gene or the expression cassette, a recombinant microorganism containing the Nat1 gene, the expression cassette or the recombinant vector, or a genetically-modified oomycete cell line containing the Nat1 gene, the expression cassette or the recombinant vector.

### CROSS-REFERENCE TO RELATED APPLICATIONS

This application is a continuation of International Patent Application No. PCT/CN2021/139229, filed on Dec. 17, 2021, which claims the benefit of priority from Chinese Patent Application No. 202110147090.7, filed on Feb. 3, 2021. The content of the aforementioned application, including any intervening amendments thereto, is incorporated herein by reference in its entirety.

### TECHNICAL FIELD

This application relates to genetic engineering, and more particularly to an application of N-cetyltransferase 1 (Nat1) gene as a screening marker in genetic transformation of an oomycete.

### BACKGROUND

Oomycetes, pertaining to eukaryotic organism, have a wide variety (at least 1800 species). Most of the oomycetes are pathogenic for plants, animals, and other organisms, posing huge economic losses. Plant pathogenic oomycetes mainly include downy mildews, Phytophthora, Pythium, and Albugo. Compared with fungi, oomycetes have some unique physiological and biochemical characteristics. For instance, hyphae of the oomycetes have no or few septa; the oomycetes have diploid vegetative parts; and cell walls of the oomycetes are mainly composed of cellulose.

Phytophthora are members of the phylum Oomycete, and are dominated by phytopathogens. Diseases caused Phytophthora, called “plant blight”, cause severe damage to many crops and plants. At present, the serious damage of diseases caused by the oomycetes to agricultural production has gradually attracted worldwide attention. Fortunately, the continuous release of genome sequence information of the oomycetes has greatly promoted the genetic research of the oomycetes, and promoted the development of new genetic manipulation tools to enable the knockout and complementation of target genes in the pathogenic oomycetes and identify their functions.

Currently, available screening markers for the oomycetes are extremely limited, and among them, NPT II gene is considered as the most reliable screening marker, which can make oomycetes resistant to geneticin (G418) by means of a protein, whose coding is promoted by the generic strong promoter ham34 the protein coding, to screen the transformant. However, when the NPT II gene is introduced to a knockout system, the transformant carrying the NPT II gene cannot be reused in the complementation due to the G418 resistance. Therefore, there is still a lack of an available screening marker for the gene complementation of knockout transformants.

There are some reports on the design and development of novel selection markers for oomycete transformation. Chinese Patent Application Publication No. 108373497A discloses an oxathiapiprolin resistance gene and an application thereof as a screening marker in the oomycete transformation, where the encoding gene of a mutated protein that can induce the oomycete to produce more than 500 times resistance to the oxathiapiprolin is used as a screening marker for screening the transformant during the transformation of Phytophthora sojae. Moreover, Chinese Patent Application Publication No. 108060173A discloses an application of a carboxylic acid amide (CAA) fungicide resistance gene as a screening marker for the oomycete transformation, where the encoding gene of a mutated protein that can induce the oomycete to produce more than 100 times resistance to the carboxylic acid amide fungicide is used as a screening marker for selecting the transformant during the transformation of the Phytophthora capsici.

Although the CAA compounds and oxathiapiprolin are pesticides for the prevention and control of oomycete diseases, the extensive use of these pesticides in the agricultural production will induce strains to develop drug resistance. Therefore, the above-mentioned screening markers are still limited in the actual application.

Therefore, the development of novel screening markers for the oomycete transformation is of great significance for the exploration of gene functions of oomycetes.

### SUMMARY

An object of the present disclosure is to provide an application of Nat1 gene as screening marker in genetic transformation of an oomycete to overcome the defects in the prior art that there is a lack of an available screening marker for the gene complementation of oomycetes. Moreover, the use of the Nat1 gene as the marker has high screening efficiency for the oomycete transformants.

Technical solutions of the present disclosure are described as follows.

In a first aspect, the present disclosure provides a selection method for genetic transformation of an oomycete, comprising:

selecting a transformant by using a Nat1 gene as a selection marker;

wherein a nucleotide sequence of the Nat1 gene is listed in SEQ ID NO: 1.

The Nat1 gene is a nourseothricin resistance gene, i.e., nourseothricin N-acetyl transferase 1. The amino acid sequence of the nourseothricin N-acetyl transferase (NAT) is shown in SEQ ID NO:2.

In an embodiment, the oomycete is a Phytophthora sojae.

It should be noted that Phytophthora sojae is used herein as a test strain for illustrating the application of the Nat1 gene in the genetic transformation of the oomycete. The Nat1 gene can also be applied as a screen marker to the genetic transformation of Phytophthora capsici, Peronophythora litchii, and other oomycetes, which are all included in the scope of this application.

In an embodiment, the oomycete transformant is a complemented mutant of a knockout transformant screened using other marker genes, or a knockout mutant of a knockout transformant screened using other marker genes.

It should be noted that the other screening markers can be NPT II gene commonly used in the prior art, but do not exclude other screening markers. The novel selection marker provided herein diversify the screening marker in the prior art. Whether the screening marker provided herein is used alone, or in combination with other screening markers to perform more complex genetic engineering experiments (i.e., multi-step knockout or complementation experiments), it all falls within the scope of the present application.

In an embodiment, the complemented mutant is screened through steps of:

(S1) constructing a complementation vector containing the Nat1 gene and a target gene;

(S2) preparing an oomycete protoplast from an oomycete with the target gene knocked out;

(S3) transferring the complementation vector to the oomycete protoplast via polyethylene glycol (PEG) mediated transformation to obtain a complemented transformant; and

(S4) culturing the complemented transformant in a nourseothricin-containing culture to screen the complemented mutant.

It should be noted that the preparation of the oomycetes with the target gene knocked out pertains to the prior art, which is not limited here. For example, in an embodiment, the target gene in the oomycete is replaced with external NPT II gene (as a selection marker) in the presence of the geneticin G418 to obtain the oomycete with the target gene knocked out (i.e., the desired knockout transformant).

In an embodiment, in step (S1), the complementation vector is constructed through steps of:

linearizing pTOR vector; and ligating the Nat1 gene into a linearized pTOR vector via a homologous recombination enzyme to obtain a recombination pTOR vector; and

linearizing the recombination pTOR vector via ClaI endonuclease and EcoRI endonuclease; and ligating the target gene into a linearized recombination pTOR vector via T4 ligase to construct the complementation vector.

In an embodiment, the step (S4) is performed through steps of:

culturing the complemented transformant in a nourseothricin-containing PM solid culture medium at 25° C. in dark for 3-4 days to perform primary screening;

when hyphae are observed, covering a layer of a nourseothricin-containing V8 solid culture medium on the PM solid culture medium followed by culture at 25° C. in dark for 3-4 days to perform secondary screening; and

transferring a regenerated strain to another nourseothricin-containing V8 solid culture medium to perform tertiary screening to obtain the complemented mutant.

In an embodiment, a concentration of nourseothricin in the nourseothricin-containing PM solid culture medium is 30-50 μg/mL to ensure the selection efficiency and positive rate of the transformant.

In a second aspect, the present disclosure provides a Nat1 gene-related biological material for genetic transformation of an oomycete, wherein the Nat1 gene-related biological material is

an expression cassette containing Nat1 gene;

a recombinant vector containing the Nat1 gene or the expression cassette;

a recombinant microorganism containing the Nat1 gene or the expression cassette or the recombinant vector; or

a genetically-modified oomycete cell line containing the Nat1 gene, the expression cassette, or the recombinant vector;

wherein a nucleotide sequence of the Nat1 gene is shown in SEQ ID NO: 1.

In an embodiment, the expression cassette refers to a DNA sequence capable of expressing the Nat1 gene in the host cell. The DNA sequence comprises not only a promoter that initiates the transcription of the Nat1 gene, but also a terminator that terminates the transcription of the Nat1 gene. The expression cassette further comprises a target gene, and the promoter and the terminator used in the target gene and the Nat1 gene are different.

In an embodiment, the promoter that initiates the transcription of the Nat1 gene is Hsp70 promoter, and its nucleotide sequence is shown in SEQ ID NO:3. The terminator that terminates the transcription of the Nat1 gene is Hsp70 terminator, and its nucleotide sequence is shown in SEQ ID NO:4. The promoter that initiates the transcription of the target gene is the Ham34 promoter, and its nucleotide sequence is shown in SEQ ID NO:5. The terminator that terminates the transcription of the target gene is Ham34 terminator, and its nucleotide sequence is shown in SEQ ID NO:6.

In some embodiments, the promoter and the terminator can be Ham34 promoter HJV and Ham34 terminator HJV; or 5′ P. sojae RPL41 promoter and 5′ P. sojae RPL41 terminator. The promoter and the terminator are not specifically limited, and as long as they can function in the gene expression in the oomycetes.

In an embodiment, the recombinant vector comprises a DNA molecule encoded with the Nat1 gene. The recombinant vector containing the Nat1 gene or the Nat1 gene expression cassette can be constructed using existing molecular cloning vectors or oomycete expression vectors, such as a pTOR vector and a pYF515 vector.

In an embodiment, the recombinant microorganism is selected from the group consisting of yeast, bacteria and fungi.

In an embodiment, the oomycete is Phytophthora sojae.

Compared to the prior art, the present disclosure has the following beneficial effects.

It has been found for the first time that the nourseothricin (NTC) can be used as a selection marker in the oomycete transformation. By using the encoding gene Nat1, which can induce the oomycetes to be resistant to nourseothricin, as a selection marker to select the transformant during the oomycete transformation, the screening efficiency of the transformant can reach 90% or more, and the labor consumption is greatly reduced. The selection marker provided herein diversifies the selection markers for the oomycete transformation and effectively supports the gene complementation and gene multi-knockout during the oomycete transformation.

The calculation of the selection efficiency is described below. The transformant selected by a culture medium containing nourseothricin is considered as the candidate transformant. The candidate transformant is subjected to DNA extraction and RNA extraction to identify whether the transformant can successfully express the target gene. Consequently, the selection efficiency is a ratio of those transformants that can successfully express the target gene to all candidate transformants.

The above description is an overview of the technical solutions of the present application, aiming to allow those skilled in the art to understand the technical solutions of the present application clearer and implement the technical solutions of the present application according to the description and the accompanying drawings. To render the objects, features, and advantages of this application more understandable, the present application will be described below with reference to the embodiments and the accompanying drawings of this application.

### BRIEF DESCRIPTION OF THE DRAWINGS

The accompanying drawings are only illustrative of the principles, implementation, application, features, and effects of the embodiments of the present application, and are not intended to limit the application.

FIG. 1 illustrates a growth of colonies of an Avh109 gene complemented transformant acquired by using Nat1 gene as a selection marker, where from left to right, the first plate and the second plate both are wild-type Phytophthora sojae (WT), and the third plate and the fourth plate both are the Avh109 gene replenishment transformant;

FIG. 2 shows PCR verification results of the Avh109 gene complemented transformant acquired by using the Nat1 gene as the selection marker, where M: Marker; WT: wild-type Phytophthora sojae; 1 and 2: the Avh109 gene complemented transformant; and PsACT is a conserved gene in the wild-type Phytophthora sojae and is used as a test control group; and

FIG. 3 shows PCR verification results of the Avh109 gene complemented transformant acquired by using the Nat1 gene as the selection marker and the expression of the Nat1 gene, where M: Marker; WT: wild-type Phytophthora sojae; KO (knockout): Avh109 gene-knockout strain; and complemented lines: Avh109 gene complemented transformant (15 candidate transformants).

### DETAILED DESCRIPTION OF EMBODIMENTS

It should be noted that the following description is merely illustrative, and are not intended to limit the disclosure. Unless otherwise specified, all technical and scientific terms used herein have the same meaning as commonly understood by those skilled in the art; and the use of related terms is only descriptive, and is not intended to limit the present application.

Phytophthora is very distinctive from fungi in classification. Phytophthora belongs to the class oomycetes of the phylum oomycota and, where its cell wall is mainly composed of β-glucan and cellulose; the hypha is septum-free and coenocytic; and the cell nucleus of the Phytophthora is diploid. While the fungi belong to the phylum fungi, and its cell wall is mainly composed of chitin, and the cell nucleus is haploid. Therefore, most of the antibiotics used for selection of the fungi cannot be applied to oomycetes. Nourseothricin is an antibiotic that can inhibit the protein synthesis, which can effectively inhibit the growth of many prokaryotes and eukaryotes. Nevertheless, it has not been used in the agricultural production yet.

It has been surprisingly discovered that the screening of oomycetes can be performed using the Nat1 gene as a selection marker in the presence of nourseothricin, and the screening process has simple operation and excellent repeatability.

To make those skilled in the art understand the technical solutions of the present application clearer, the disclosure will be described in detail below with reference to embodiments.

Unless otherwise specified, the experimental materials used herein are available commercially.

The enzymolysis buffer solution is prepared from 0.15 g of lsing enzyme, 0.06 g of celluse, 10 mL of 0.8M mannitol solution, 8 mL of ddH₂O, 800 μL of 0.5M KCl solution, 800 μL of 0.5M 2-(N-morpholino)ethanesulfonic acid (MES), and 400 μL of 0.5M CaCl₂) solution.

The 40% PEG solution is prepared from 6 g of PEG400, 3.75 mL of 0.8M mannitol, 3 mL of 0.5M CaCl₂), and 3 mL of H₂O.

The W5 solution is prepared by dissolving 0.186 g of KCl, 9.2 g of CaCl₂)—H₂O, 4.5 g of NaCl, and 15.6 g of glucose with ddH₂O (to a final volume of 500 mL).

The 0.8M mannitol solution is prepared by diluting 145.76 g of mannitol to 1 L with ddH₂O.

The 0.5 M CaCl₂) solution is prepared by dissolving 14.7 g of CaCl₂)—H₂O with ddH₂O to a final volume of 200 mL.

The 0.5 M KCl solution is prepared by dissolving 7.5 g of KCl with ddH₂O to a final volume of 200 mL.

The MMg solution is prepared by dissolving 18.22 g of mannitol, 0.76 g of MgCl₂-6H₂O and 2 mL of 0.5 M MES with ddH₂O to 250 mL.

The nutritious pea-based medium (NPB) is prepared through as follows. 60 g of peas are put into a conical flask, to which an appropriate amount of deionized water is added. The conical flask is sterilized at 121° C. for 15 min and filtered to obtain a filtrate. After that, the filtrate is added with 0.5 g of KH₂PO₄, 0.65 g of K₂HPO₄.2H₂O, 1.5 g of KNOB, 0.25 g of MgSO₄, 0.04 g of CaCl₂, 1.0 g of CaCO₃, 2.5 g of D-sorbitol, 2.5 g of D-glucose, and 1 g of Yeast Extract in sequence, dissolved, centrifuged at 4000×g for 10 min, diluted to 500 mL, and sterilized at 121° C. for 15 min.

The PM solid medium is prepared as follows. 60 g of peas are put into a conical flask, to which an appropriate amount of deionized water is added. The conical flask is sterilized at 121° C. for 15 min and filtered to obtain a filtrate. After that, the filtrate is added with 45.5 g of Mannitol, dissolved, centrifuged at 4000×g for 10 min, diluted to 500 mL, added with 1.0 wt % agar, and sterilized at 121° C. for 15 min.

The V8 solid medium is prepared through the following steps. The V8 vegetable juice (available commercially) is added with 0.2% CaCO₃ powder, dissolved and centrifuged at 5000 rpm for 20 min to obtain a supernatant. The supernatant is diluted with 9 times volume of deionized water, added with 1.5-2.0 wt % agar, and sterilized at 121° C. for 15 min.

### Example 1 Application of Nat1 Gene as Screening Marker in the Gene Complementation of Phytophthora sojae

1. Cloning of Nat1 Gene and Target Gene (Avh109 Gene)

The Nat1 gene was cloned using the primer pair Nat1-F and Nat1-R, and the nucleotide sequence of the cloned Nat1 gene was shown in SEQ ID NO:1.

Nat1-F:
(shown in SEQ ID NO: 7)
5′-ACACAAGGGCCCCGTTTCGCAT
GGGTACCACTCTTGACG-3′;
and
Nat1-R:
(shown in SEQ ID NO: 8)
5′-TTCGAACCCCAGAGTCCCGC
TTAGGGGCAGGGCATGCT-3′.

The Avh109 gene was searched from the NCBI database (http://www.ncbi.nlm.nih.gov/), and its CDS and genome sequence was downloaded to design primers G Avh109-F and G Avh109-R to clone the Avh109 gene fragment, and the nucleotide sequence of the cloned Avh109 gene was shown in SEQ ID NO:9.

G Avh109-F:
(shown in SEQ ID NO: 10)
5′-CCATCGATATGCGTCTCCAGTATGCCG-3′;
and
G Avh109-R:
(shown in SEQ ID NO: 11)
5′-GGAATTCATCAGCGGTTTGTCGCC-3′.

2. Ligation of Target Gene (Avh109 Gene) and Nat1 Gene into a Complementation Vector pTOR

The pTOR was linearized by using linearized vector primers (pTOR-F and pTOR-R), and then the Nat1 gene was ligated into the pTOR vector by using a homologous recombination enzyme to obtain a recombinant vector, which was subjected to sequencing to determine whether the pTOR vector had been successfully constructed. The recombinant vector was linearized by using endonucleases ClaI and EcoRI, and ligated with the Avh109 gene through the T4 ligase to obtain the complementation vector, which was confirmed by sequencing. The nucleotide sequences of the pTOR-F and pTOR-R were respectively shown as follows:

PTOR-F:
(SEQ ID NO: 12)
5′-GCGGGACTCTGGGGTTCG-3′;
and
PTOR-R:
(SEQ ID NO: 13)
5′-GCGAAACGGGGCCCTTGT-3′.

The pTOR-R vector contained universal promoters and terminators for oomycetes, where the Hsp70 promoter (SEQ ID NO:3) and the Hsp70 terminator (SEQ ID NO:4) regulated the expression of the Nat1 gene; and the Ham34 promoter (SEQ ID NO:5) and the Ham34 terminator (SEQ ID NO:6) regulated the expression of the Avh109 gene.

3. The complementation vector was transformed to a Phytophthora protoplast via a PEG-mediated transformation system to obtain a transformant, which was specifically described below.

(1) Preparation of Experimental Materials

A wide-type Phytophthora sojae strain P6497 was activated on a nutritious pea-based medium (NPB) plate, and cultured at 25° C. in dark (the activated hyphae were used within one week). It shall be noted that the wide-type Phytophthora sojae strain P6497 was only the material used herein, and actually, any commercially available oomycete strains were applicable to the screening method provided herein.

After cultured for 3-4 days, the hyphae of the wide-type Phytophthora sojae strain P6497 were cut into a plurality of mycelial pellets with a size of 3*3 mm. Three 250 mL Erlenmeyer flasks containing 50 mL of the NPB medium was each added with six mycelial pellets, and subjected to stationary culture at 25° C. in the dark for 2.5-3 days, where the Erlenmeyer flask was shaken once a day during the culture.

The hypha of the cultured wide-type Phytophthora sojae P6497 was used as the parent to obtain an Avh109-knockout transformant, in which the Avh109 gene was replaced with the exogenous gene NPT II by using a common gene knockout tool in the art, such as the method proposed by Yufeng et al. (Yufeng Fang, Linkai Cui, et al. Efficient Genome Editing in the Oomycete Phytophthora sojae Using CRISPR/Cas9[J]. Current Protocols in Microbiology, 2017).

(2) Preparation of Protoplasts

(S1) An ultra-clean bench was subjected to UV sterilization for 30 min. After that, a lyase and 40% PEG were added into a sterilized 50 mL beaker, and this operation should be strictly regulated to reduce pollution.

(S2) A sterilized 200 mL beaker covered with gauze in advance was used to collect the hyphae (i.e., the Avh109 gene-knockout transformant). The hyphae were fed to a 50 mL centrifuge tube containing 40 mL of 0.8 M mannitol solution by using a pair of forceps and washed once. Then the hyphae were collected to the sterilized 200 mL beaker. After that, the hyphae were transferred to a 50 mL centrifuge tube containing 35 mL of 0.8 M mannitol buffer with a pair of forceps, sealed, mixed, and shaken at room temperature and 60 rpm for 10 min.

(S3) An enzymolysis buffer was prepared as follows. Ingredients with specified volumes were added into a sterilized 50 mL beaker, stirred with a pipette tip for full dissolution and filtered by a biofilter to collect the enzymolysis buffer in a 50 mL centrifuge tube.

(S4) The hyphae were washed with the 0.8 M mannitol buffer, collected and added into the enzymolysis buffer to undergo an enzymolysis reaction at 25° C. and 60 rpm for 1-1.5 h, where the adding amount of the hyphae was not allowed to be excessive.

(S5) The 40% PEG solution was prepared as follows. The ingredients with specified volumes were dissolved completely, filtered with the biofilter and placed on ice for use.

(S6) A refrigerated centrifuge was started.

(S7) After the enzymolysis reaction, the hyphae were filtered to collect the protoplasts by using two layers of mira-cloth (imported filter cloth). The protoplast lysis was observed under a microscope. Then the collected protoplasts were transferred to a 50 mL centrifuge tube to be centrifuged at 4° C. and 1500 rpm for 3 min. In the subsequent operations, the protoplasts needed to be kept at a low temperature.

(S8) After the centrifugation, the supernatant was discarded. Then about 10 mL of W5 solution were added to gently resuspend the protoplasts. The W5 solution was added to 35 mL to be centrifuged at 4° C. and 1500 rpm for 4 min, and the condition of the protoplasts was observed under the microscope.

(S9) After the centrifugation, the supernatant was discarded. Then about 10 mL of W5 solution were added to gently resuspend the protoplasts. The concentration of the protoplast was calculated by using a blood count plate, stood on ice for 30 min, and centrifuged at 4° C. and 1500 rpm for 4 min.

(S10) After the centrifugation, the supernatant was discarded. Then a pre-cooled MMg solution was added to resuspend the protoplasts. The concentration of the protoplast was adjusted to 2×10⁶/mL, followed by standing at the room temperature for 10 min.

The transformation of the protoplasts was described below.

(S1) A 50 mL centrifuge tube was placed on an ice, and added with 30 μg transformation plasmids (namely, a complementation vector including the Nat1 gene and the Avh109 gene).

(S2) 1 mL of protoplasts were added into the 50 mL centrifuge tube, mixed gently, and stood on the ice for 5-10 min.

(S3) 1.74 mL of PEG solution was divided into three parts and successively added into the 50 mL centrifuge tube, followed by gentle mixing, and standing on the ice for 20 min.

(S4) A cephalexin antibiotic was added into a pre-cooled PM culture medium for use.

(S5) 2 mL of the pre-cooled PM culture medium added with the cephalexin antibiotic in step (S4) were added into the 50 mL centrifuge tube, followed by gentle mixing, and standing on the ice for 2 min.

(S6) 8 mL of the pre-cooled PM culture medium added with the cephalexin antibiotic in step (S4) were added into the 50 mL centrifuge tube, followed by gentle mixing, and standing on the ice for 2 min.

(S7) The 50 mL centrifuge tube was subjected to standing at 25° C. for 12-14 h to obtain a culture (namely, a transformant), where the 50 mL centrifuge tube was inclinedly placed to increase the contact area with oxygen.

The complemented mutant of the Phytophthora knockout transformant was screened through the following steps.

(S1) 5 μL of the culture was taken to observe a regeneration protoplast via the microscope and, and then the culture was centrifuged at 2000 rpm for 5 min to obtain a regenerated hypha.

(S2) After the centrifugation, the supernatant was discarded. Then 5 mL of PM culture medium were added into the centrifuge tube to resuspend the regenerated hypha. After that, 5 mL of 30 μg/mL nourseothricin-containing PM culture medium were added into the centrifuge tube, and mixed to be poured into a petri dish. Then the petri dish was subjected to drying, and culturing at 25° C. in dark for 3-4 days to perform primary screening.

(S3) The growth condition of hyphae on the surface of the petri dish was observed. 10 mL of 50 μg/mL nourseothricin-containing V8 culture medium were added into the petri dish, and cultured at 25° C. in the dark for 3-4 days to perform secondary screening.

(S4) The growth condition of hyphae on the surface of the petri dish was observed. The regenerated strain was transferred to the 50 μg/mL nourseothricin-containing V8 culture medium to perform tertiary screening. After three screenings in the presence of nourseothricin, 15 transformants were obtained, which were preliminarily considered to be complemented transformants (candidate transformants) for subsequent identification.

### Example 2 Verification of Phytophthora Complemented Mutant

(S1) Verification of Transformants by Using a Nourseothricin-Containing Culture Medium

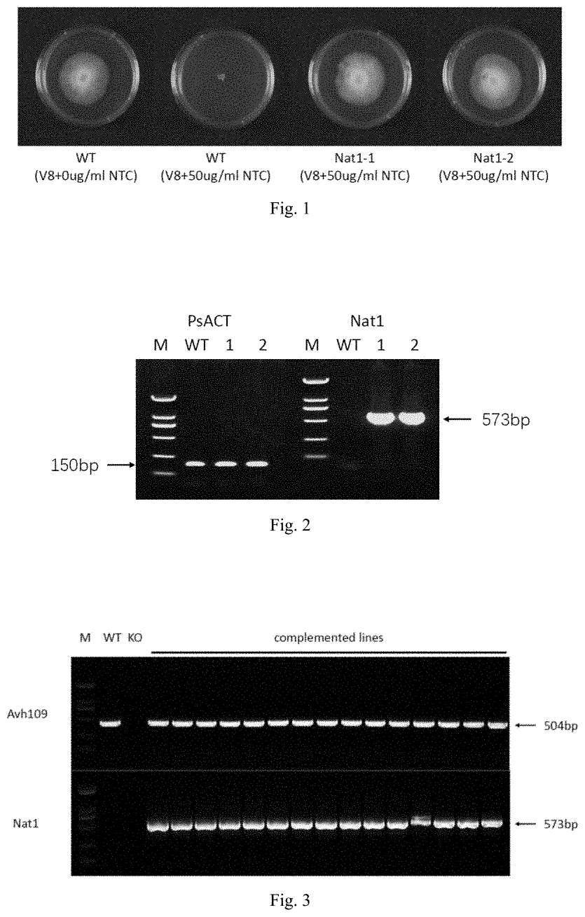

The 15 candidate transformants screened in Example 1 were transformed to the 50 μg/mL nourseothricin-containing V8 culture medium to observe the growth condition of the 15 candidate transformants, and the wide-type Phytophthora sojae is used as a control strain. As shown in FIG. 1, the screened candidate transformants were able to grow normally on the 50 μg/mL nourseothricin-containing V8 medium (only 2 of the candidate transformants were shown in FIG. 1, and the growth situation of the remaining 13 candidate transformants was the same as the 2 candidate transformants illustrated in FIG. 1). On the contrary, the control strain was not able to grow on 50 μg/mL nourseothricin-containing V8 medium, while was capable of growing on the V8 medium without nourseothricin.

(S2) DNA Extraction of Candidate Transformants for PCR Verification

Genomes of the wide-type Phytophthora sojae and the complemented transformant (candidate transformant) were extracted via a cetyltrimethylammonium bromide (CTAB) method. The Nat1 gene was subjected to PCR amplification, and the wide-type Phytophthora sojae was used as control. The verification results were shown in FIG. 2, in which the wide-type Phytophthora sojae was free of the Nat1 gene, while the complemented transformant contained the Nat1 gene (the PsACT gene in FIG. 2 was a conserved gene of the wide-type Phytophthora sojae and was used as control to demonstrate the reliability of the genome extraction.

(S3) RNA Extraction of Transformant for Detection of Gene Expression

RNA of the Avh109 gene-knockout strain (KO), the wide-type strain (WT) and the 15 complemented transformants was extracted via a RNA extraction kit, and subjected to reverse transcription to obtain cDNA after the DNA was removed. After that, the cDNA was used as a template to verify expression of the target Avh109 gene through RT-PCR. The expression results were shown in FIG. 3, from which it can be observed that the KO strain failed to express the Avh109 gene, while the WT strain and the screened complemented transformants expressed the Avh109 gene successfully. Calculated by three replicate tests, the screen efficiency of the complemented transformants could reach more than 90%.

The results showed that the positive transformants (candidate transformants) selected in Example 1 all successfully expressed the Avh109 gene. It was demonstrated through the statistical analysis that the complemented transformant could be screened with an efficiency more than 90% when using the Nat1 gene as the screening marker in the presence of nourseothricin, overcoming the lack of screening marker in the genetic transformation of oomycetes in the prior art.

The calculation of the screening efficiency was described below. The transformant screened by a nourseothricin-containing culture medium was considered as the candidate transformant. The candidate transformant was subjected to DNA extraction and RNA extraction to identify the transformant that successfully expressed the target gene. Consequently, the screening efficiency was a ratio of the transformant that successfully expressed the target gene to the candidate transformant.

It should be noted that described above are only preferred embodiments of the present disclosure, which are not intended to limit the present disclosure. When the technical parameters that are not described in detail are changed within the range listed herein, the same or similar technical effects as the above-mentioned embodiments can still be obtained, which still fall within the scope of the disclosure. It should be understood that any modifications, replacements, and improvements made by those skilled in the art without departing from the spirit of this application shall fall within the scope of this application defined by the appended claims.

What is claimed is:

1. A method for screening an oomycete transformant, comprising:
employing Nat1 gene as a screening marker to screen the oomycete transformant;
wherein a nucleotide sequence of the Nat1 gene is shown in SEQ ID NO: 1.

2. The method of claim 1, wherein the oomycete transformant is derived from Phytophthora sojae.

3. The method of claim 1, wherein the oomycete transformant is a complemented mutant of a knockout transformant screened using other marker genes, or a knockout mutant of a knockout transformant screened using other marker genes.

4. The method of claim 3, wherein the complemented mutant is screened through steps of:
(S1) constructing a complementation vector containing the Nat1 gene and a target gene;
(S2) preparing an oomycete protoplast from an oomycete with the target gene knocked out;
(S3) transferring the complementation vector to the oomycete protoplast via polyethylene glycol (PEG)-mediated transformation to obtain a complemented transformant; and
(S4) culturing the complemented transformant in a nourseothricin-containing culture medium to screen the complemented mutant.

5. The method of claim 4, wherein in step (S1), the complementation vector is constructed through steps of:
linearizing a pTOR vector; and ligating the Nat1 gene into a linearized pTOR vector via a homologous recombination enzyme to obtain a recombination pTOR vector; and
linearizing the recombination pTOR vector via ClaI endonuclease and EcoRI endonuclease; and ligating the target gene into a linearized recombination pTOR vector via T4 ligase to construct the complementation vector.

6. The method of claim 4, wherein the step (S4) is performed through steps of:
culturing the complemented transformant in a nourseothricin-containing PM solid culture medium at 25° C. in dark for 3-4 days to perform primary screening;
when hyphae are observed, covering a layer of a nourseothricin-containing V8 solid culture medium on the PM solid culture medium followed by culture at 25° C. in dark for 3-4 days to perform secondary screening; and
transferring a regenerated strain to another nourseothricin-containing V8 solid culture medium to perform tertiary screening to obtain the complemented mutant.

7. The method of claim 6, wherein a concentration of nourseothricin in the nourseothricin-containing PM solid culture medium is 30-50 μg/mL.

8. A Nat1 gene-related biological material for genetic transformation of an oomycete, wherein the Nat1 gene-related biological material is an expression cassette containing Nat1 gene;
a recombinant vector containing the Nat1 gene or the expression cassette;
a recombinant microorganism containing the Nat1 gene, the expression cassette or the recombinant vector; or
a genetically-modified oomycete cell line containing the Nat1 gene, the expression cassette, or the recombinant vector;
wherein a nucleotide sequence of the Nat1 gene is shown in SEQ ID NO: 1.

9. The Nat1 gene-related biological material of claim 8, wherein the genetically-modified oomycete cell line is derived from Phytophthora sojae.


SEQUENCE LISTING (13 sequences)

SEQ ID NO: 1 - DNA, 573 bp, Artificial Sequence
  synthetic
atgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactctcggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggtgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaa

SEQ ID NO: 2 - protein, 190 aa, Artificial Sequence
  synthetic
MGTTLDDTAYRYRTSVPGDAEAIEALDGSFTTDTVFRVTATGDGFTLREVPVDPPLTKVFPDDESDDESDDGEDGDPDSRTFVAYGDDGDLAGFVVVSYSGWNRRLTVEDIEVAPEHRGHGVGRALMGLATEFARERGAGHLWLEVTNVNAPAIHAYRRMGFTLCGLDTALYDGTASDGEQALYMSMPCP

SEQ ID NO: 3 - DNA, 584 bp, Artificial Sequence
  synthetic
cgtcaatgcaattgactgtgtcctcgattatttctattattaatattatttccattaaaaaagaaatccagttcgtggcaaaataagacaaaggtttaaataaaaatttatcgttattaaactcgtaacttaccttcttcatggctcaacccaatctgtatatcgacttatttaatttggttaacaaatcggttttcgtcgcaaataggggatgacaactaaaataccaatttgtgtccttgcgacacgtcaacaggtggctgccaatatctcaggttttgagcacacaacgctgtgattggctatctgtagatcgcttcgagaactttcggaccgacatatcagaacgcagtcatgtccaatccagctaacgattggtcgaaaccgcacttcatcgcttcatttcaaccaattcccatctactaaaacgagcgtcggaatagtctcgaacgtaaccccacgtagtgggtggcagcgcccctcgtatctttcaatcgccactcgagtctcaagtttgctcactttgaaattttccatctggactacacatcaaatctaactcaacttttatcaattgaaacaca

SEQ ID NO: 4 - DNA, 220 bp, Artificial Sequence
  synthetic
gtcgactagaatacttagcaattggttagtttgaaaattgtcaaagcagagttttttcgacaataaaatttattttctaaatatttgatttagcgttgacttaatttgttgattcggttgtcatcttttgtgaagatgcccgagagtctagtaaatctcaagcgaaacatagtgagaaggtcactatggctggtttgcatgagtctgggcctattgtgct

SEQ ID NO: 5 - DNA, 565 bp, Artificial Sequence
  synthetic
tctgatggacaaagggtcgcctctacgtacccgcgacaatctcggcttttccaatgtcgacagctggcatatcgcacgcgaggttaggcgtcttcagccggcgtaaccacaaacctcgagtcaagtaccctttttcagaccttaatttccgtgtccattccctcgcgtttacacgaaggtttctgagaaataccacatgtcacagagagagagaagattggagaaccttctcaacctgacccgtgcagagcgcttttgcgtcctaccatccgttaattatagcaccaccaccacatgtctcgccggaactctcttatccgctctttttgatcgtcttttttgcgccccattaaaagacatctcttgcacagggatcgtttctgattggccatttcacttcctcttttacatccgaaaaccacctgtttgtggggttgtgaagccacaaccggacgctcaaaaagcctcgcccgactcgcccacgatcggaaggctcattctccttttcactctcacgtgcccaagtcccaaccgactctttttcgaccttcactctcaccgacaa

SEQ ID NO: 6 - DNA, 477 bp, Artificial Sequence
  synthetic
tgtgtctatgcatttaatacaatttccttttttccattttcgttgtttttttttgtgaaagaacgattgttttcaaaagaaggccgtttgtagaacaatttttttttatactagcatttggtattcgagtgtgacgaatcggtgatggcgctttacaaatgaaatataacgctgaacaagcgcagtgacagctttattttataacagagaataaagaccaaagtagagtagactgatggcggcttgaaacaaaactacgtgacttctaatgcaaagaaaaccataccccacgctacttagaattgtttctctttggttgatgaccgctatagtcgtatggagtgatccctttttgtcaatatttcgattcttgctaaaaccgtgactattttgaggcaactgtgtgtacttaacatggtagaaatttctatgtttaagccagctgacaatcattctttatttatcgtaaaccaacca

SEQ ID NO: 7 - DNA, 39 bp, Artificial Sequence
  synthetic
acacaagggccccgtttcgcatgggtaccactcttgacg

SEQ ID NO: 8 - DNA, 38 bp, Artificial Sequence
  synthetic
ttcgaaccccagagtcccgcttaggggcagggcatgct

SEQ ID NO: 9 - DNA, 504 bp, Artificial Sequence
  synthetic
atgcgtctccagtatgccgtagtcgtggctgctgctgctctggccgcgagctccgaaggacttcaggtcctccccaattcggccaagtccacgtcgctgcgagcaagcgctgaagctcggtacccgcctttcgtcgagggcaagggagaccgcttcttggttagcgaaggcaatcaggaatggtcgactcagacccagaagggctacgagttctcgccgctgcaggagcaggacgacgtgctgcagcagaacgacgatgagtacgaagacgacagcgactcgagctcacagagcgagagcggcttcgatgacgaggccaggctcttcgggaggaagaagaagaagaagaaaaagaagaaacacgaagccaccgagacaccaacgcccacaccgacggcgactccagagggaatgaccgcgactcccaccccagcgcctactacggaggagccaagcggctggaggaaattccttgcctggtacaggcgacaaaccgctgattaa

SEQ ID NO: 10 - DNA, 27 bp, Artificial Sequence
  synthetic
ccatcgatatgcgtctccagtatgccg

SEQ ID NO: 11 - DNA, 24 bp, Artificial Sequence
  synthetic
ggaattcatcagcggtttgtcgcc

SEQ ID NO: 12 - DNA, 18 bp, Artificial Sequence
  synthetic
gcgggactctggggttcg

SEQ ID NO: 13 - DNA, 18 bp, Artificial Sequence
  synthetic
gcgaaacggggcccttgt